(19) United States
(12) Patent Application Publication
(10) Pub. No.: US 2024/0067993 A1
(43) Pub. Date: Feb. 29, 2024

(54) METHOD OF CULTURE

(71) Applicant: NORWEGIAN UNIVERSITY OF LIFE SCIENCES, Ås (NO)

(72) Inventors: Lars Reier BAKKEN, Ås (NO); Linda BERGAUST, Ås (NO); Svein Jarle HORN, Ås (NO)

(73) Assignee: NORWEGIAN UNIVERSITY OF LIFE SCIENCES, Ås (NO)

(21) Appl. No.: 18/270,538

(22) PCT Filed: Jan. 5, 2022
(86) PCT No.: PCT/EP2022/050170

§ 371 Date: Jun. 30, 2023

(30) Foreign Application Priority Data
Jan. 5, 2021 (GB) 2100105.2

(51) Int. Cl. C12P 1/04; C12N 1/20

(52) U.S. Cl. CPC C12P 1/04; C12N 1/20

(57) ABSTRACT

The present invention provides a method for anaerobic culture of denitrifying microorganisms in culture medium in which nitric acid and/or nitrous acid are added two or more times to the medium during culture, and in which the pH of the medium is maintained within a pH interval by the addition of the nitric acid and/or nitrous acid and the nitric acid and/or nitrous acid also provides the electron acceptor for denitrification in the culture. Methods of producing a cell culture product using such methods and the products which are obtained are also provided.

The present invention relates to a method for anaerobic culture of a denitrifying microorganism in culture medium using nitric acid and/or nitrous acid to provide electron acceptors for respiratory metabolism and to maintain the culture within a pre-determined pH interval for optimum growth. This method allows growth to a high cell density and has particular utility in producing cell culture products such as single cell proteins (SCPs) or specialist molecules from the microorganisms which may be used, for example, in food or in pharmaceuticals, respectively.

High cell density cultivation (HCDC) is a powerful technique for the production of desirable products such as recombinant proteins, antibiotics and single cell microbial proteins. The annual market growth for recombinant proteins is expected to increase at a rate of 10-15% per annum (Shojaosadati et al., 2008, Iranian J. Biotech., 6(2), p63-84). Single cell microbial proteins are considered to be a potential solution to the growing demand for protein, e.g. as a feed and food source (Ritala et al., 2017, Frontier in Microbiology, 8, p1-18). There is a particularly compelling case for growth of chemoautotrophs which generate biomass by fixing CO₂ using H₂ and O₂ as electron donor and acceptor, respectively, and therefore produce protein “from thin air” (Matassa et al., 2015, Water Res., 68, p467-478; Matassa et al., 2016, Water Research, 101, 137-146). Bacteria offer a particularly attractive feed- and food-source as they can contain up to 70% dry weight of protein of high nutritional value (Volova & Barashkov, 2010, Appl. Biochem. Microbiol., 46(6), p574-579).

HCDC also faces a number of density- and upscaling-related challenges, e.g. high metabolic rates require optimization of mixing and gas (if relevant) and nutrient supply, and nutrients must be continuously or intermittently fed to the culture in carefully determined proportions to avoid toxic effects. Also, at high density, oxygen deprived and/or fermenting cultures run the risk of accumulating metabolites (e.g. organic acids) to inhibitory concentrations. Moreover, hidden auxotrophies may limit growth (e.g. Humphrey, 1998, Biotech. Progress, 14(1), p3-7). To date, high density cultivation of bacteria has taken advantage of chemoautotrophic growth, heterotrophic aerobic respiration or fermentation, each with their challenges and limitations.

Traditionally, single cell microbial proteins are produced by aerobic respiration in aerated fermenters (Ritala et al., 2017, supra). However, aeration (provision of oxygen) is a significant cost in such production, but it is also limiting on the production rate (Shojaosadati et al., 2008, supra). To illustrate: at biomass density of 20 g L⁻¹, the potential O₂ consumption rate at growth rate 0.2 h⁻¹ is ˜3 mmol L⁻¹ min⁻¹, while the solubility of O₂ is ˜0.2 mmol L⁻¹ (equilibrium with air at 35° C.). This implies that the oxygen pool has a turnover rate >15 min⁻¹. This is the approximate maximum oxygen supply rate that can be achieved in conventional laboratory scale fermenters (Mare et al, 2005, Biotechnology Letters, 27, p983-990), albeit with high costs and technical challenges such as foaming, flooding and shear stress. Higher oxygen supply rates can be achieved by using pure oxygen, high pressure, or dialysis (Subramaniam et al, 2018, Chem. and Biochem. Eng. Quart., 32(4), p451-464), although the cost efficiency of these measures is questionable. Methods of anaerobic culture using denitrifying bacteria are known (such as in WO00/29604). However, these are not suitable for high cell density cultivation. It has been found by the inventors that such methods result in high salt accumulation during culture at high cell densities which inhibits cell growth.

There is therefore a need for alternative methods of high cell density cultivation, particularly for microorganisms that are able to produce desirable products but for which no suitable high cell density cultivation methods are available.

To date, only aerobic methods of high cell density cultivation by respiration have been pursued. As far as the inventors are aware, no methods of anaerobic culturing to high cell densities are known.

The present inventors have developed an anaerobic method of cultivation using denitrifying microorganisms which allows high cell density cell cultivation and avoids the limitations of aerobic cultivation, minimizes the costs of aeration, and eliminates the problem of salt accumulation that would occur if the electron acceptors were provided as salts.

Denitrification is the most energy yielding form of anaerobic respiration and involves the dissimilatory reduction of nitrate (NO₃) to N₂ via nitrite (NO₂⁻), nitric oxide (NO) and nitrous oxide (N₂O). The process is not limited to specific taxa or lifestyles, although many belong to the proteobacteria and the majority depend solely on respiration, i.e. do not ferment. Denitrifiers are typically facultative anaerobes, which prefer O₂ as electron acceptor, and they proliferate in systems where oxygen concentration fluctuates, be it soil, water, sediments or animals. They are heterotrophs or autotrophs, mesophiles or extremophiles and they are the major biological source and only known sink for N₂O(Shlesinger, 2009, PNAS, 106(1), p203-208); the third most important greenhouse gas and dominant destroyer of stratospheric ozone (Denman et al., 2007, Report on the Intergovernmental Panel on Climate Change, pp499-587; Ravishakara et al., 2009, Science, 326(5949), p123-125). The biochemistry and physiology of denitrification have been studied in a selection of model organisms (e.g. Bergaust et al., 2008, Environ. Microbiol., 10(11), p3070-3081; Bergaust et al., 2010, Appl. Environ. Microbiol., 76(19), p6387-6396; Liu et al., 2013, Environ. Microbiol., 15(10), p2816-2828) and their role in nitrogen loss from agricultural soil and application in wastewater treatment is well described. Recently, their potential within mitigation of N₂O emission has been explored, e.g. through selection of N₂O reducing inoculants in biofertilizers (e.g. Akiyama et al., 2016, Nat. Publ. Gr. 6:32869.doi:10.1038/srep32869; Woliy et al., 2019, Frontiers in Microbiol., 10, p2746) or as part of other emerging soil improvement strategies.

Whilst anaerobic cultivation of denitrifying microorganisms is known, no known methods are suitable for high cell density cultivation.

In anaerobic respiration which occurs during denitrification, nitrate (NO₃⁻), nitrite (NO₂⁻), nitric oxide (NO) and nitrous oxide (N₂O) act as the electron acceptors, replacing O₂. Known methods of culture use salts such as KNO₃ to provide the nitrate. Nitrate reduction leads to alkalization, which during HCDC would need to be counteracted by addition of acids (i.e. HCl), eventually resulting in accumulation of M concentrations of salt and growth arrest. For example, given the growth yield per mol NO₃ for anaerobic respiration (14 g cell dry weight per mol NO₃⁻), the salt concentration would reach inhibitory levels (>0.5 M, Canovas et al., 2007, Biotechn. Bioeng., 96, p722-737, doi: 10.1002/bit.21128) as the culture reaches a density of >7 g cell dry-weight L⁻¹).

It has now surprisingly been found that anaerobic culture is possible in which high cell density can be achieved by denitrification. The present inventors have developed a method in which the accumulation of inhibitory salt levels can be avoided allowing culture to be continued to high cell densities. The inventors have identified that nitrate may be provided in the form of strong (˜5 M) nitric acid, which does not result in salt accumulation during culture. Preferably, the nitric acid is used as the rate-limiting component in the culture method. Nitric acid could be replaced by a mixture of nitric and nitrous acid or nitrous acid alone (which may similarly be rate-limiting). To achieve optimal growth the nitric acid and/or nitrous acid is added to the culture during the culture process. The timing of its addition is tied to the pH of the culture medium. Consumption of the nitric acid/nitrous acid leads to an increased pH. As this occurs additional nitric acid/nitrous acid can be added to both lower the pH and provide more of the electron acceptor. Thus, the concentration of the electron acceptor is controlled by pH-homeostasis using nitric acid and/or nitrous acid to counteract the metabolic alkalinisation which occurs by the reduction of NO₃⁻ to N₂.

The advantage of this method is that the flow rate of the nitric/nitrous acid into the system controls the metabolic rate of the system (its concentration can be kept well above rate-timing concentrations so as to secure maximum growth rate, or within a rate-limiting concentration range). The addition of the acid also allows the pH to be controlled, even at high production rates. Furthermore, no salt build-up, which is detrimental to cell growth, occurs. Another advantage is that NO₃ can be assimilated, thus sustaining protein production on substrates (carbon sources) that contain little nitrogen (such as sugar hydrolysates from lignocellulose-rich plant material, methane etc.). This is optional, however; because the microorganisms will assimilate NH₄⁺ if provided in sufficient amounts with the culture feed.

These high cell density methods have the advantage that they reduce costs (as oxygen is not required) and allow high production rates per volume (not currently possible in aerobic methods in which oxygen is a limiting factor).

Thus, in a first aspect the present invention provides a method for anaerobic culture of denitrifying microorganisms in culture medium, comprising the addition of nitric acid and/or nitrous acid two or more times to said medium during said culture, wherein the pH of the medium is maintained within a pH interval by the addition of the nitric acid and/or nitrous acid.

The essence of the invention is that nitric/nitrous acid is used as an electron acceptor in the anaerobic respiration method and is added repeatedly during the culture method. This avoids salt build-up that would occur if other sources of nitrate were used and further serves to maintain pH levels at optimal levels during respiration as well as nitrate concentrations at a given level (based on feedback from the pH levels).

As defined herein, “culture” refers to an in vitro method in which cells (in this case microorganisms) are grown in a culture medium over a period of time under conditions appropriate to sustain their viability. Such conditions include appropriate pH, temperature, osmolality, gas concentration and using a medium containing essential components necessary for growth.

As referred to herein, viability refers to microorganisms that remain alive and able to replicate.

The “culture medium” is the liquid medium in which the cells are grown. This contains essential components for growth. Typically this includes: an energy/carbon source, usually in the form of a saccharide such as glucose, all essential amino acids, vitamins, free fatty acids, inorganic salts, trace elements (usually in the micromolar range), for example, which are necessary for growth and/or survival. The solution may also contain components that enhance growth, or induce or repress the production of specific metabolites of interest. The solution is preferably optimized to an appropriate pH and salt concentration suitable for cell survival and proliferation and a buffer may be used to maintain pH (e.g. HEPES), and to secure the desired relationship between the concentration of NO₃⁻ and pH.

In cell culture involving denitrification an electron donor and electron acceptor is also required. The former may be provided by the carbon source. The latter is provided by nitric acid/nitrous acid which is added to the medium. Thus, in accordance with the invention, the pH of the medium is maintained within a pH interval by the addition of the nitric acid and/or nitrous acid and said nitric acid and/or nitrous acid also provides the electron acceptor for denitrification (which occurs during the anaerobic culture of denitrifying microorganisms) in said culture. In particular the nitric acid/nitrous acid provides the electron acceptor for anaerobic respiration in the denitrification method.

The nitric acid and/or nitrous acid in this method counteracts the alkalinizing effect of the consumption of nitrogen oxyanions (nitrate and nitrite) by the microorganisms, e.g. bacteria. This pH control, by addition of nitric acid and/or nitrous acid, has a dual role, securing both acceptable pH and provision of electron acceptors to sustain the anaerobic respiration and growth. This contrasts with conventional cultivation of denitrifying microorganisms, such as bacteria, where the primary source of electron acceptors are nitrate- and/or nitrite salts. In preferred aspects the nitric acid and/or nitrous acid provides the sole electron acceptor for anaerobic respiration (denitrification) in the culture, thereby securing provision of electron acceptors according to demand since the alkalinizing effect of the respiration is counteracted by injection of more acid(s).

Thus, in a particular aspect the invention provides a method for anaerobic culture of denitrifying microorganisms in culture medium, comprising the addition of nitric acid and/or nitrous acid two or more times to said medium during said culture, wherein the pH of the medium is maintained within a pH interval by the addition of the nitric acid and/or nitrous acid and said nitric acid and/or nitrous acid addition also provides the electron acceptors for denitrification in said culture, wherein said addition compensates for the nitrate which has been consumed during said culture. As referred to herein the addition of nitric acid and/or nitrous acid compensates for the nitrate by providing sufficient additional nitrate to sustain further culture without the need for additional nitrate from a different molecule, e.g. without the need to add a nitrate salt.

A defined or natural medium or mix thereof may be used. Additions may be made to the medium during culture (=culture feed), as discussed hereinafter, to provide components necessary for sustained growth to high cell densities, i.e. to provide a perfusion culture or fed-batch culture. Appropriate media/culture feed for growth may be selected according to the microorganism to be grown. Depending on the microorganism, suitable media formulations include commercially available media such as Ham's F20 (Sigma), Minimal Essential Medium (MEM, Sigma), RPMI-1640 (Sigma) and Dulbecco's Modified Eagle's Medium (DMEM, Sigma). For growth of bacterial cells, Sistrom's medium with succinate and ammonium (Bergaust et al., 2008, Environ. Microbiol., 10, p3070-3081; Bergaust et al., 2010, Appl. Environ. Microbiol., 76(19), p 6387-6396) may be used. Medium as described by Hahnke (2014, Front. Microbiol., 5(18), p1-9) may also be used. Media as described in the Examples may also be used. The requirements of the microorganism to be used should be taken into account in selecting the medium. Depending on the microorganism, suitable formulations for the medium and culture feed components can be made to suit the microorganism's requirement for an electron donor and carbon source, specific organic compounds that the microorganism is unable to synthesize (=auxotrophy), and suitable concentrations of macro elements (P, K, Mg etc.) and microelements (Fe, Zn, Mn, Mo, Ni etc).

The microorganisms are preferably grown in suspension. Preferably, the suspension is agitated, e.g. stirred, during culture to allow the medium to access the cells evenly.

The culture is conveniently performed in a bioreactor which is a vessel suitable for growth of cells. Conventional bioreactors can include fed-batch stirred reactors, batch stirred reactors or continuous flow stirred reactors. The bioreactor is of a suitable size to allow cell growth, preferably to a high density. The bioreactor contains a vessel in which the cell culture is performed. For example, the bioreactor vessel has a capacity of at least 1 liter, preferably at least 100, 1000, 10,000 liters or more. Suitable bioreactors are well known and include bioreactors such as autoclavable glass fermenters (for example from Applikon) or stainless steel fermenters (for example from Biolaffite). The internal conditions of the bioreactor vessel, including, but not limited to pH and temperature, are typically controlled during the culturing period.

The bioreactor also comprises inlets and outlets to the vessel in which culture occurs which are suitable for addition (and optionally extraction) of reagents held in reservoirs (or material produced during the culture process), e.g. an inlet for nitric acid and/or nitrous acid addition. Pumps to allow the passage of liquids into and out of the bioreactor vessel may also be provided. The bioreactor may also be provided with suitable monitoring means, e.g. a pH monitor to assess the pH of the culture medium during culture and/or monitors for other gases such as CO₂. The operation of the bioreactor may be controlled by an operating system (e.g. a computer) which receives input from sensors on the status of the culture (e.g. pH, temperature, levels of starting materials, by-products or products), processes that information, and sends instructions to controlling means, such as pumps or temperature controlling means, to modify the culture conditions accordingly.

“Anaerobic” culture refers to culture without oxygen, i.e. under anoxic conditions. In methods of the invention strict anoxia may not be needed, however, and depending on the microorganism's requirement, a minimum of oxygen provision may be desirable, although the major respiratory electron flow is channelled to denitrification.

“Denitrifying microorganisms” are microorganisms which use denitrification to generate proton motive force and ultimately ATP. During this anaerobic respiratory process, nitrogen (N) oxides replace oxygen as terminal electron acceptors, and are converted as follows: NO₃⁻→NO₂⁻→NO→N₂O→N₂, using nitrate reductase (Nar or Nap), nitrite reductase (Nir), nitric oxide reductase (Nor), and nitrous oxide reductase (Nos) for these conversions, respectively. Several denitrifying microorganisms can use both oxygen and nitrate as electron acceptors at the same time.

The reactions that occur may be summarized as follows:

Conversion of nitrate to nitrite by nitrate reductase (Nar):

2NO₃⁻+4H⁺+4e⁻→2NO₂+2H₂O

Conversion of nitrite to nitric oxide by nitrite reductase (Nir):

2 NO₂⁻+4H⁺+2e⁻→2 NO+2H₂O

Conversion of nitric oxide to nitrous oxide by nitric oxide reductase (Nor):

2NO+2H⁺+2e⁻→N₂O+H₂O

Conversion of nitrous oxide to dinitrogen by nitrous oxide reductase (Nos):

N₂O+2H⁺+2e⁻→N₂+H₂O

The source of H⁺ is NADH for all reactions except the nitrite reduction, where 2H⁺ are from NADH and 2H⁺ are protons from the medium. This is the alkalinizing reaction. Thus, the reduction of 1 mol NO₃ to % N₂ consumes 1 mol H⁺ from the medium, and hence increases the pH.

Denitrifying microorganisms encompassed herein express the enzymes Nar, Nir and Nor. They may additionally express the enzyme Nos, though this is not essential. Such microorganisms include bacteria, fungi and archaea. The ability to denitrify is present in a wide range of species. Nevertheless, preferably the fungi are from the genera Fusarium, Gibberella, Nectria, Cylindrocarpon, or Trichoderma. Preferred fungi also include yeast from the genera Candida, Saccharomycopsis or Trichosporon. Preferred archaea include archaea from the genera Haloarcula or Haloferax.

In a preferred aspect the denitrifying microorganisms are bacteria. Preferably, the bacteria are from the genera Paracoccus, Pseudomonas, Alkaligenes, Bacillus or Streptomyces. Especially preferably the bacterium is Paracoccus denitrificans or Pseudomonas stutzeri. Appropriate microorganisms can be identified by determining whether they express the required denitrification enzymes.

As referred to herein, microorganisms are referred to interchangeably as cells, e.g. when referring to the production of products of culture.

Depending on the desired products of culture, the denitrifying microorganism may be selected accordingly. Thus, for example, if the cell culture product of interest is the microorganisms themselves, e.g. for use in feed, then the microorganism may be selected based on desirable nutritional properties of the microorganism, e.g. high protein levels and low levels of undesirable molecules. When the cell culture product is a molecule generated by the microorganisms, the microorganism is selected or modified to maximize production of that molecule. For example, a microorganism may be genetically modified to encode a protein of interest which is then produced by the microorganism.

The microorganism is appropriately selected to ensure that high cell density culture can be tolerated, e.g. to avoid microorganisms that may release inhibiting compounds at high densities or which release quorum sensing compounds which alters the phenotype in an undesirable way. This may, to some extent, be overcome by the appropriate selection of the carbon source.

In the method of the invention, nitric acid and/or nitrous acid is added two or more times to the culture medium during the culture. In the method the nitric acid and/or nitrous acid acts as an electron acceptor, i.e. it is an oxidizing agent that is reduced in a redox (reduction-oxidation) reaction.

Nitric acid is readily available (e.g. Sigma Aldrich). The nitric acid to be added to the culture medium may be provided at a high concentration for the purposes of addition, e.g. from 1-10M (preferably 4-8M) or ≥2M or ≥5M, e.g. 2-15, 5-15, 2-15 or 5-15 M. Nitrous acid is less effective than nitric acid as an electron acceptor, but a mixture of nitric and nitrous acid may be desirable for specific microorganisms or applications. In some cases, nitrous acid may be used alone and may be used at the same concentrations as nitric acid. Nitrous acid is available commercially or may be provided in mixtures with nitric acid (for example in the ratio of nitric acid:nitrous acid of 100:1 to 1:1, e.g. 10:1 to 5:1). For example, nitrous acid or a mixture of nitric and nitrous acid (for example in a 10:1 ratio) can be produced by the plasma-technology developed by N₂Applied, Norway, (https://n2applied.com/). However, in a preferred aspect nitric acid is used alone or in combination with nitrous acid.

As referred to herein “addition” refers to active administration of the entity of interest (e.g. nitric acid/nitrous acid, carbon source, trace elements) to the culture medium. Any convenient means may be used, e.g. manual addition or addition with a pump (including automated methods reliant on detection of pH levels with a probe, as described in more detail hereinafter). In the case of nitric acid and/or nitrous acid, it may be added directly to the culture medium as a liquid in the required concentration, e.g. as described above.

The addition of nitric acid and/or nitrous acid is performed to maintain the pH of the culture medium within a pH interval. As noted above, during anaerobic culture of denitrifying microorganisms the pH increases as the reaction progresses and the nitrate which is present in the medium is consumed. The addition of nitric acid and/or nitrous acid reduces the pH and thus can be used to maintain the pH of the medium in a pH interval as well as providing more nitrate to compensate for the nitrate which has been consumed. The amount and timing of the addition is dictated by the pH of the culture medium but the acid (nitric and/or nitrous) may be added to an initial concentration of 2 to 10 mM in the culture at the point of each addition. Thus the nitrate deriving from nitric acid and/or nitrous acid may have this initial concentration.

The addition may be performed two or more times. The number of times for the addition to be made depends on the length of the culture and the conditions of the culture. Short culture may only require a small number of additions to maintain the pH interval whereas a lengthy culture may require more additions. A larger number of additions may be necessary if a smaller pH interval is to be maintained or the culture conditions are such that the pH is changing rapidly (e.g. based on cell density). Preferably the addition is conducted at least 5, 10, 20, 50, 100, 200, 500, 1,000, 5,000, 10,000 or more times, e.g. up to 100, 200, 500, 1,000, 5,000, 10,000 or 20,000 times or within a range defined by the minimum and maximum times described herein, e.g. from 10-200 times.

The additions are made to control pH and thus should follow, and be responsive to, the pH changes during culture (which result from alkalinisation by anaerobic respiration). As such, the additions are not necessarily made at regular intervals or with the same amount each time, but rather the timing and amounts are selected based on the pH change that is necessary to maintain the pH interval. Thus the addition may be at various intervals (which may be regular or irregular, e.g. in response to monitoring) or continuously. The result of irregular provision is that the pH will vary during the reaction, but this is acceptable providing it is maintained within the defined pH range which ensures that the desired ratio between the different components is maintained.

Nevertheless, the nitric acid and/or nitrous acid is preferably added at least every hour during said culture, preferably at least every 30, 20, 10, 5, 2 or 1 minute(s). Especially preferably the nitric acid and/or nitrous acid is added to the medium continuously or semi-continuously during said culture.

The pH interval to be maintained in the method of the invention is determined according to the microorganism to be cultured. The pH interval provides a range over which optimal/maximal growth is achieved for that microorganism. Optimal growth refers to rapid division of the cells, preferably exponential growth. The determination of the growth cycle for the microorganism can be determined for the particular microorganism by methods known to those in the art. The optimal pH (which may be a single point or a range, and is the pH over which near-maximum growth rates (optimal growth) are achieved) may be readily determined by assessing growth of the microorganism of interest in the culture medium and under the conditions to be used for its growth. The microorganism's optimal pH may, for example, be anywhere between pH 5 and 8. For Paracoccus denitrificans, for example, the optimal pH is 7-7.5. The pH interval to be maintained encompasses the optimal pH or where the optimal pH is a range, at least part of that optimal pH range. A narrow pH interval will ensure optimized growth. Thus in a preferred aspect, the pH interval is ≤1 pH unit, preferably 50.5, 0.4, 0.2, 0.1 or 0.05 pH unit. By way of example, if the optimal pH is 7.4, the pH interval may be 0.2 of a pH unit and may be from 7.3-7.5.

As noted above the pH interval is selected according to the microorganism under culture. Conveniently, the pH interval may be from pH 7 to 8 or a smaller interval within the range, but this depends entirely on the microorganism. For Paracoccus denitrificans, for example, the pH interval may be 7.1-7.7 or 7.3-7.5.

Within the pH interval is the midrange. The midrange is the mean of the lower and upper ends of the pH interval. In the pH interval 7.3-7.5 the midrange is 7.4. The midrange and the optimal pH may be coincident, but this is not necessarily required.

In a preferred aspect, the midrange of the pH interval is within +/−0.4 pH units (preferably within +/−0.2 or 0.1 pH units) of the microorganism's optimal pH range, or midpoint of that range. The midrange of the pH interval is preferably in the range of pH 7 to 8, though this will vary depending on the optimal pH for the microorganism in use.

In order to maintain the pH interval, if the pH rises above the midrange point, nitric acid and/or nitrous acid should be added to maintain the pH range. Thus, in a preferred aspect, the nitric acid and/or nitrous acid is added to the medium when the pH of the medium rises above the midrange of the pH interval, for example when the pH rises to the upper level of the pH interval. However, it is advisable to add nitric acid and/or nitrous acid before the upper level of the pH interval is reached to avoid the culture exceeding the bounds of the pH interval and to keep the pH of the culture as close to the optimal pH (or at least midrange pH) as possible.

In order to make additions in response to pH changes, conveniently the pH of the culture medium is monitored during the reaction e.g. as described in the Examples. Suitable pH probes are known in the art (e.g. EasyFerm Plus PHI Arc. 225, Hamilton; digital pH sensor Memosens CPS171D, Endress & Hauser; AppliSens pH+ sensor, Applikon). For example the pH may be monitored regularly or irregularly throughout the culture method, e.g. 2 or more, e.g. 3, 5, 10 or more times, e.g. up to 50 or 100 times, preferably at least every 30, 20, 10, 5, 2 or 1 minute(s). Conveniently, monitoring may be conducted continuously. Thus in a preferred aspect, the pH of the culture medium is monitored and nitric acid and/or nitrous acid is added when the pH of the medium rises above the midrange of the pH interval, or when the pH rises to the upper level of the pH interval.

Whilst control of the pH is key to providing an effective method, other parameters may be assessed or monitored during the culture period. For example, depending on the conditions used, other entities that are consumed or produced during the culture may be assessed or monitored. This may include assessing or monitoring of one or more products or starting materials in the denitrification process, e.g. nitrate, nitrite, nitric oxide, nitrous oxide or nitrogen gas. Similarly, levels of other gases which are involved in the process (e.g. consumed or produced) may be assessed or monitored, e.g. O₂, CO, CH₄, H₂ or CO₂. The culture medium may also be assessed or monitored for the presence of other essential components, e.g. essential trace elements such as trace metals. Furthermore, the build-up or presence of damaging or unnecessary molecules during the culture method may be assessed or monitored, e.g. alcohols and organic acids. The growth or cell density may also be monitored, either continuously in the reactor (e.g. by backscatter or NIR probes) or offline by e.g. by OD₆₆₀ measured by a spectrophotometer. Yield may also be assessed based on cell dry weight L⁻¹.

For performance of the method of the invention, an electron donor, electron acceptor, carbon and nitrogen source are required. The electron acceptors of the denitrification method are nitrate, NO₃⁻, nitrite, NO₂⁻, nitric oxide, NO, and N₂O. Nitrate can also be utilized as a nitrogen source, if the microorganism has the ability to assimilate nitrate. If not, ammonium can be added as a nitrogen source. Nitrate is added in the form of nitric acid and/or nitrous acid by addition during the culture method, as discussed hereinbefore. However, in addition, nitrates may be provided in the starting culture medium or other additions made during culture. These nitrates may be provided by the addition of nitric acid and/or nitrous acid or by the use of nitrate salts, e.g. KNO₃ or Co(NO₃)₂. However, the use of such salts should be limited to avoid the build-up of salts during the culture method. In one alternative, no nitrate salts are used in the culture method. Particular preferably, nitric acid and/or nitrous acid is used as the sole electron acceptor in the culture method. However, on occasion nitrate salts may be used, but in a preferred aspect no nitrate salts are used in the method of the invention except in the initial starting culture. Nitrate salts can be used in the starting culture to avoid an impact on pH (which would result from the use of nitric/nitrous acid) before the culture method has commenced. When nitrate salts are used they may be used as an initial small dose (e.g. 1-10 mM), sustaining the initial growth to typically 1-10 mg cell dry-weight L⁻¹, while the subsequent cultivation to high cell densities (>10 g cell dry-weight L⁻¹) is preferably sustained by the addition (e.g. stepwise) of nitric acid and/or nitrous acid only.

In a preferred aspect the nitric acid and/or nitrous acid provides at least 50% of the nitrate to the culture, e.g. at least 60, 70, 80, 90 or 95% of the nitrate.

As noted, herein, the method of the invention serves to avoid accumulation of salt which has inhibitory effects on cell growth. Thus, the salt concentration in said culture should not reach inhibitory levels for the organism cultured. Whilst some salt may be tolerated in the culture method, in a preferred aspect, the salt concentration in said culture resulting from the addition of nitrate to said culture is less than 1M, preferably less than 750 mM, 500, 400, 300, 200 or 100 mM. This is particularly relevant for culture at high cell density, e.g. based on cell dry weight L⁻¹ of the culture, e.g. greater than 3, 5, 7, 10, 15, 20 or more cell dry weight L⁻¹.

The salt results from the addition of nitrate in the form XNO₃, wherein X is a cation (that is not a non-metal cation consumed during the culture), preferably a metal cation such as Na⁺, K⁺ or Ca²⁺. Nitrate reduction leads to alkalization, which during high cell density culture needs to be counteracted by the addition of an acid (HY, where Y is an anion such as Cl⁻ or SO₄⁻), eventually resulting in accumulation of the salt XY, and hence growth arrest. As noted previously this may reach 0.5 M as the cell density reaches 7 g cell dry-weight L⁻¹, which is sufficient to cause severely retarded growth of microorganisms such as bacteria. As referred to herein the concentration of salt is therefore the concentration of XY that is present in the culture resulting from the addition of the nitrate and acid. There may be other salts present in the culture, e.g. from the use of trace elements, but the amount present would be negligible and may therefore be ignored in calculating the salt concentration which is calculated as described above.

The carbon source may provide, or allow, the generation of the electron donor. Alternatively a separate electron donor and carbon source may be used. The selection of carbon source/electron donor may reflect the microorganisms that are used. One carbon source may be used or a mixture of carbon sources may be used. The denitrifying microorganisms that may be used include heterotrophs (using organic carbon as a food source), autotrophs (e.g. using C1 compounds such as CO₂, CH₃OH or CH₄ as the carbon source, and a suitable electron donor, for example CH₄, H₂, CO or formate), mesophiles (which grow at moderate temperatures) and extremophiles (that grow under extreme conditions of e.g. temperature or pH). Autotrophs include chemoautotrophs, such as hydrogenotrophs which grow on H₂ and CO₂ or other C1 carbon sources (in which in the method of the invention H₂ provides the electron donor and CO₂ the carbon source) and methanotrophs (in which CH₄ provides the electron donor and organic carbon source). Mixotrophs which use inorganic sources of energy and organic carbon may also be used. In preferred aspects of the invention, mixotrophic cultivation using H₂ as an electron donor in addition to the carbon source is used.

In one preferred aspect, the denitrifying microorganism is an autotrophic microorganism, and preferably the carbon source for said culture is provided by CO₂ and the electron donor for said culture is provided by hydrogen.

In preferred methods, heterotrophs are used in which the carbon source (which may also provide the electron donor) is a saccharide or the base of an organic acid (e.g. formate, acetate, propionate, butyrate or succinate, which may be provided in an appropriate non-ionic form to the medium). Suitable saccharides include monosaccharides such as such as glucose, fructose, xylose, xylulose, arabinose, mannose, galactose, or disaccharides such as sucrose, maltose or lactose. More complex oligo- or poly-saccharides may also be used such as cellulose or starch (e.g. with from 3 to 100 (or more) monomers). Preferably the carbon source is glucose. Appropriate concentrations may be determined based on the culture conditions. The ratio between the rate of addition of the carbon source and the nitric/nitrous acid is dictated by the product stoichiometry of the growth by the microorganism used (i.e. XNO₃+YC→Z biomass-C, in which X, Y and Z in this case refer to stoichiometric amounts and do not relate to the salt concentration described hereinbefore), and the concentration of the carbon source and the concentration of HNO₃ (and/or nitrous acid) that is added, e.g. from respective reservoirs. For example, monosaccharides such as glucose may be provided by addition to the culture medium to a concentration of 5 to 30 mM, for example.

The culture method may be performed over a short period of time, but is preferably conducted over long period of times. Indeed, the method of the invention is intended to allow extended culture times and high cell density culture. Thus, in a preferred aspect the culture is performed for at least 50 hours, preferably at least 100, 200 or 300 hours, e.g. up to 20, 30 or 100 days. In a particularly preferred aspect, the culture in continuous (as described hereinafter in more detail) and culture of upwards of 30 days may be performed.

The method described herein may form only part of a cell culture method (or may form the full culture method from the start of the culture to harvest). Thus, for example, the culture could begin under aerobic conditions but then move to anaerobic conditions as oxygen is depleted. In such a case the method of the invention commences only once anaerobic denitrifying culture according to the method of the invention begins. However, it is possible, depending on the microorganism, that a minimum of O₂ provision, be it as pulses or as continuous flow, is desirable, although the main respiratory electron flow goes to denitrification.

In another alternative the method may begin at low cell density at which some salt accumulation (or other conditions) may be tolerated and then move to high cell density during culture. The method of the invention may commence at the start of that culture or only once the culture reaches high cell density as defined herein.

As discussed above, the method of the invention allows microorganisms to be grown to a high cell density, particularly by avoiding the build-up of harmful/toxic substances and providing optimized conditions for growth in terms of pH and reagents required for growth. As referred to herein, “cell density” refers to the number of cells (i.e. individual microorganisms) in a unit of volume. In a preferred aspect, the microorganisms in the culture are grown to a high cell density (providing a high cell density culture), for example at least 1×10¹³ cells L⁻¹, preferably at least 1×10¹⁴ cells L⁻¹. In an alternative, high cell density may be assessed based on the cell dry weight of microorganisms obtained from the cell culture. In a preferred aspect, the cell dry weight is at least 3 g, 5 g or 7 g cell dry weight L⁻¹ preferably the cell dry weight is at least 10 g, 15 g or 20 g cell dry weight L⁻¹, for example at least 30 or 50 g cell dry weight L⁻¹, for example up to 100 g cell dry weight L⁻¹. Cell dry weight may be assessed as set out in Example 1. Cultures are preferably grown to such a cell density. In some aspects culture may commence at such a cell density.

In addition to controlling the rate of the reaction and the pH by the addition of nitric acid and/or nitrous acid, other elements of the process may be controlled to further optimize the method. Thus, in a further preferred aspect of the invention, the partial pressure of CO₂ (P_{CO2}) is controlled within an acceptable concentration range in the culture headspace and hence in the medium. The limits for the acceptable range is dictated by the microorganism's tolerance to P_{CO2}, but also by the effect of P_{CO2} on pH. High P_{CO2} lowers the pH, hence it affects the relationship between pH and the concentration of NO. When CO₂ is the carbon source (autotrophic growth) in the method, P_{CO2} must be sustained above a minimum. In such cases addition of CO₂, e.g. by injection, may be required.

The “culture headspace” refers to the volume of the bioreactor vessel in which the microorganisms are cultured excluding the volume occupied by the culture medium. P_{CO2} may be lowered by sparging the culture headspace or medium with another gas, preferably a gas which does not interfere with the culture conditions, e.g. N₂, helium or argon. As an alternative, the culture may be sparged by the headspace gas, circulated via a CO₂ trap. Preferably CO₂ is monitored during the process and removed as necessary to maintain P_{CO2} within an acceptable range. Where CO₂ is not required during culture (e.g. for heterotrophic organisms) substantially all of the CO₂ may be removed from the culture headspace during the culture method. As referred to herein “substantially all” refers to the extent of removal possible by conventional methods.

The repeated addition of nitric acid and/or nitrous acid in response to an increasing pH reflects consumption (reduction) of the electron acceptor and is an indicator that respiration is occurring. In line with the consumption of the electron acceptor, the electron donor (which may be provided by the carbon source) is similarly consumed (or in the case of the electron donor, oxidized), as well as the carbon source. To allow culture and cell growth to continue, not only the electron acceptor, but also the carbon source (and optionally a separate electron donor) needs to be added to the culture medium to avoid it becoming rate-limiting in the culture method. Additional components, such as macro- and micro elements (P, K Mg, Mn, Zn, Cu, etc) may also be provided to sustain cell growth.

Thus, in a preferred aspect, the carbon source (and optionally a separate electron donor) and/or trace elements are added to the medium two or more times during said culture, wherein preferably the carbon source (and optionally a separate electron donor) and trace elements are added separately. As discussed above, there are certain instances when a separate electron donor is used, e.g. H₂ for hydrogenotrophic growth. In this instance, H₂ gas may be provided to the culture headspace multiple times (as discussed above) or continuously. This provision of H₂ is not in conflict with continuous removal of CO₂ if this is achieved by circulation of the culture headspace via a CO₂-trap and back to the culture via the sparger. This will also secure transport of H₂ into the liquid.

The number and timing of additions of the carbon source (and optionally a separate electron donor) and/or trace elements may be as set out above for the nitric acid and/or nitrous acid. The additions may be coincident with the additions of nitric acid and/or nitrous acid or may be performed at different times. Nevertheless, to ensure that growth of the microorganisms can continue in the most optimal fashion, these additions are linked to those of the nitric acid/nitrous acid. In particular, it is preferable that the nitrate provided by nitric acid/nitrous acid, i.e. an electron acceptor, is the rate-limiting component for growth of the microorganisms in the culture method. It may be provided within a rate-limiting concentration range or well above the rate-limiting concentration.

Thus, in this preferred aspect, the carbon source (and optionally a separate electron donor) and/or trace elements should be present in the culture methods, at all times, at levels such that they are not rate-limiting on the growth of the microorganisms, but not at high levels that would adversely affect that growth.

As referred to herein a component which is “rate-limiting” is limiting on the rate of growth of the microorganisms as it appears at a sub-stoichiometric concentration to allow respiration. When present in a rate-limiting amount, an increase in that component's concentration will increase the rate of growth. Once an increase in the rate of growth no longer occurs when the components' concentration is increased, that component is no longer in a rate-limiting amount. The need for an addition of the carbon source may be determined based on various parameters of the culture method which may be monitored, i.e. based on the pH, the amount of nitric acid/nitrous acid added, the amount of a product produced or the cell density, for example.

Thus, in a preferred aspect, during the culture method of the invention, the carbon source (and optionally a separate electron donor) is added to the medium in an amount and at a time, relative to the amount and timing of the nitric acid/nitrous acid additions, so that the concentration of the carbon source is not rate-limiting on growth of the microorganism. This may apply to the full culture method or only a part thereof, e.g. growth before very high cell densities are achieved. Preferably the carbon source is added in a proportional amount to the nitric acid/nitrous acid, so that their relative amounts remain essentially constant.

In this preferred aspect, it may be assumed that unrestricted growth is the target (i.e. the concentrations of all elements are sustained above rate-limiting concentrations). At very high cell densities, however, reduced metabolic activity may be desirable, and this can be achieved by using a rate-limiting concentration of one or several elements. The choice of rate-limiting factor(s) (e.g. the electron donor or the electron acceptor) depends on the physiological reactions of the microorganism to such limitations. Thus, in an alternative aspect, the carbon source may be rate-limiting on the method, i.e. the carbon source is provided at amounts which control cell growth. In that case the other required components are not rate-limiting, i.e. are provided in a concentration which is not rate-limiting. The carbon source may be provided within a rate-limiting concentration range or well above the rate-limiting concentration. The culture method may be conducted in different phases in which different components are rate-limiting in the different phases, e.g. the nitric/nitrous acid is rate-limiting in the initial growth phase, but the carbon source is rate-limiting in the later, high density growth phase.

The same applies to any trace elements or separate electron donor that may be required (i.e. they may be present in amounts that are not rate-limiting or may be used as the rate-limiting component). The timing of their addition may be the same as or different to the timing of the nitric acid/nitrous acid and each other. Thus, all additions made to the culture may be made simultaneously or separately relative to one another. Appropriate control of the additions of the different components required for cell growth is required to avoid the accumulation of toxic intermediates of the denitrification process e.g. NO or NO₂⁻, or the presence of an inhibitory concentration of trace elements, e.g. trace elements such as Fe²⁺. This can be avoided by control of the method as discussed above, e.g. with regular addition of the required components, when needed, to maintain a low steady state concentration of NO₃⁻ and avoid potentially toxic levels of trace elements. NO, NO₂ (and N₂O) should be maintained at levels below the inhibitory concentration for the microorganism that is used to provide an effective method. This may be, for example, <0.1 μM NO and <5 mM NO₂⁻ in the medium. An assessment of a particular microorganism's requirements may be carried out as illustrated in the Examples.

An appropriate temperature for conducting the culture will depend on the microorganism, but may be in the range of 30-35° C., for example, or ±2° C. of the microorganism's temperature optimum for growth.

Different types of culture methods may be used, particularly taking into account the product to be produced. Appropriate and preferred culture methods include fed-batch or continuous cultures.

A “fed-batch” culture refers to a method of culturing cells in which additional components are added to the culture after the start of the culture process, particularly components that have been depleted. In the methods of the invention, an electron acceptor (in the form of nitric/nitrous acid) is necessarily added at least twice to the culture. In addition, as noted above, additional components may be, and are preferably, added to the culture, such as the carbon source, to ensure that growth can continue. A fed-batch culture is typically halted at some point and the cells/microorganisms or their products isolated and optionally purified. However, a fed-batch culture may be sustained by harvesting culture volume, e.g. 50-90% at intervals (repeated fed-batch)

In a continuous culture, a steady rate of growth is achieved in a constant volume over an extended period of time. This requires the continued input of required components (as discussed above) and an equal rate of removal of generated product and by-products as well as culture medium/microorganisms to sustain a constant environment with a constant volume and number of microorganisms.

The method of the invention may be used to produce a cell culture product. Thus in a further preferred aspect the present invention provides a method for producing a cell culture product comprising:
a) culturing denitrifying microorganisms by anaerobic culture using a method as described hereinbefore under conditions that allow the generation of said cell culture product; and
b) obtaining said cell culture product from said culture.

The cells referred to in this method are the microorganisms used in the culture method of the invention. A “cell culture product” is any product that is generated in the method of the invention as a result of the growth of the microorganisms. In a preferred aspect, the cell culture product is cultured cells, i.e. the microorganisms that have grown during the culture method are the product of the method.

In an alternative aspect, the cell culture product may be one or more molecules produced by the microorganisms during their culture. Such molecules include lipids, carbohydrates, polyhydroxyalkanoates, proteins and gases (such as gases that are generated during denitrification, e.g. N₂O), for example. In a particularly preferred aspect the molecule produced is a protein. The protein may be a protein naturally produced by the microorganism or the microorganism may be genetically modified to produce the protein. In a preferred aspect, the microorganism is genetically modified to express a protein of interest (which may result from expression of a heterologous gene encoding the protein or genetic modification to increase expression of an endogenous protein) and the product is a recombinant protein, e.g. an antibody, an enzyme, or a growth factor.

In another preferred alternative, the product is one that is sensitive to oxygen, i.e. for which anaerobic methods of production are advantageous.

Conditions of culture that allow the generation of a product of interest will largely depend on the product to be generated. For example, if the microorganisms themselves are the products of interest then methods that optimize cell growth are suitable. For methods in which a particular product is of interest, the culture method may be modified accordingly to maximize generation of that product, e.g. to provide an inducer of a promoter upstream of the gene encoding the protein product.

The cell culture product is obtained from the culture. When the microorganisms are the cell culture product this may be performed by collection of the culture medium containing the microorganisms and removal of the liquid culture medium to retain the microorganisms, e.g. by filtration or centrifugation. When the product is a product produced by the cell this may be obtained by any convenient means depending on the location of the product. For products secreted into the medium the medium may be collected to obtain the product. For products secreted into the culture headspace (i.e. gases), they may be collected during the culture method. For products which are not secreted into the medium, the microorganisms may be collected and treated to release the product of interest, e.g. by lysis. The obtained product may be retained in the form in which it has been obtained, as a collection of cells, or the liquour of the culture or a cell extract, or a gas, or may be further treated as described below.

In a preferred aspect, the step of obtaining the product comprises separating the product from the culture, medium and/or the microorganisms (or parts thereof, e.g. in cell lysates) and optionally further purifying the product. When the product is the microorganism themselves and the medium has been removed, no further separation is required. When the product is a gas, separation from other gases may be required. When the product is present in the medium or in a microorganism cell lysate further separation and optionally purification may be desirable. Depending on the product obtained and its context (e.g. medium or cell lysate), the product may be separated from the medium/microorganisms (or parts thereof) by an appropriate method, e.g. filtration, centrifugation, affinity methods, chromatography, precipitation etc. By way of example, single cell protein may be separated from the microorganisms by extracting protein from the microorganisms.

The separated product may be further purified, e.g. using the above described separation means, e.g. in combination, to remove any contaminants. In a preferred aspect, the product is purified to the extent that it provides >40%, e.g. >50, 60, 70, 80 or 90%, w/w dry weight in the purified preparation.

The cell culture products which are obtained by the above described methods may be further processed to provide a form with a suitable commercial use. Thus, in a further preferred aspect the method additionally comprises the step of processing the obtained (e.g. separated and optionally additionally purified) cell culture product to provide a processed cell culture product. As referred to herein, processing refers to methods of further manipulation of the product beyond simple separation and or isolation, e.g. to provide the product in a different form or to add further components to the product to generate a composition. The resulting products are referred to as processed cell culture products. By way of example, the processing step may be to dry the cell culture product and/or to formulate the cell culture product into a particular formulation or composition.

Preferred processed cell culture products include single cell protein, feed and pharmaceutical compositions. To generate single cell protein, the protein biomass separated from the microorganisms may be dried. As described above additional components may be added to provide a composition. This composition may be nutritional, e.g. feed, which could be generated by adding single cell protein to other nutritional components. Alternatively, the pharmaceutical composition may be generated by providing an obtained cell culture product in a relevant, pharmaceutically acceptable, carrier. This may be appropriate when the cell culture product is a protein. In the alternative the processing may simply be to alter the formulation of the obtained cell culture product, e.g. pressed into tablets, pellets or particles, e.g. for feed.

The invention further extends to a cell culture product or processed cell culture product obtained by a method as described hereinbefore.

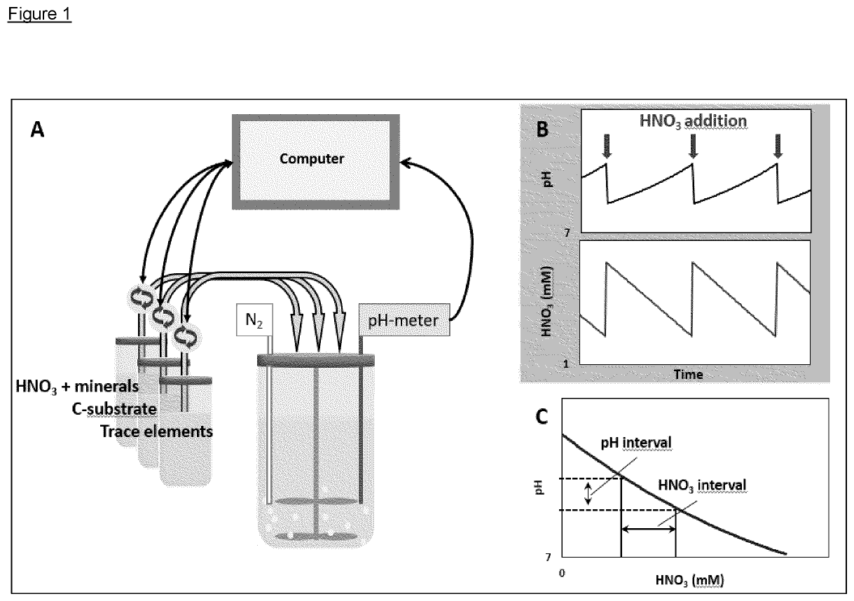

By way of example, the following method describes how the present invention may be put into effect. FIG. 1 illustrates in general terms how the invention may be put into practice.

Denitrifying microorganisms, preferably bacteria, are selected based on the desired cell culture product. Appropriate cell culture medium and culture conditions are selected based on the microorganism to be cultured. Similarly, a pH interval is selected for operation. These parameters may be determined in pre-testing before commencing the culture as set forth in the Examples.

A bioreactor is provided with a vessel and inlets and outlets. The inlets allow for the separate introduction of nitric acid/nitrous acid, the carbon source and optionally trace elements. One or more pump is provided to pump these components into the vessel from reservoirs containing the relevant components. An inlet/outlet may also be provided to allow entry of gases via a gas sparger into the culture medium or headspace, either as a flow-through (exit via the culture headspace), or by circulation of the culture headspace gas via a gas sparger. The outlet(s) is set up to allow removal of produced gases, by-products or products. A pH monitor is set up to assess the pH in the vessel during culture. An operating system is provided to receive information on the pH from the pH monitor and to activate the pump to introduce required components based on that information. Whilst the culture method may be manually adjusted to allow performance of the method, this system allows automation of the culture method.

To initiate the culture, the culture medium and a seed amount (inoculum) of microorganisms are added to the bioreactor vessel. Ideally, the culture medium is added first and brought to appropriate conditions (e.g. temperature) for culture before the microorganisms are added.

The culture method may begin with aerobic culture if convenient to do so, allowing the microorganisms to grow by aerobic respiration until the oxygen is depleted. Alternatively, and preferably, anaerobic culture may be commenced immediately. In a particularly preferred aspect, the microorganisms are precultured to transition from oxic to anoxic conditions such that the seed culture has already transitioned to, and is suitable for, anaerobic culture. In some cases, small amounts of oxygen may be advantageous for some denitrifying microorganisms. This may be achieved using sporadic manual injections or an airflow regulated by an oxygen sensor in the liquid so that the oxygen concentration is maintained at an appropriate level in the culture medium.

Once culture has commenced, pH monitoring is initiated and the operating system regulates the input of nitric acid/nitrous acid and other components from the reservoirs via the pumps to maintain the pH within a pre-set range and provide all components to the culture in non-rate-limiting (or rate-limiting) amounts as appropriate. The nitric acid/nitrous acid is provided as necessary and preferably forms the rate-limiting component of the culture method. If desired, the ratio between the rates of HNO₃ and the carbon source (which may act as the electron donor) can be changed so as to induce limitations, either electron donor- or electron-acceptor-limitations.

Cell growth and density is monitored during the culture period. In a batch-fed culture, once the desired cell density is reached the culture method is terminated and the cell culture product is harvested (optionally purified) and optionally processed. In the alternative, by harvesting only a fraction of the volume, cultivation can be continued.

The methods described in the Examples form further preferred aspects of the invention. The invention also extends to methods and products substantially as described herein with reference to the accompanying examples and drawings. All combinations of the preferred features described above are contemplated, particularly as described in the Examples. The invention will now be described by way of non-limiting Examples with reference to the drawings in which:

FIG. 1 provides an illustration of how the method of the invention (in one embodiment) may be performed. The stirred culture volume receives HNO₃, C-substrate, trace elements and minerals (either from individual reservoirs, or as one mix), the feed rate is controlled by measured pH, ensuring that pH is kept within a narrow interval. The respiration of NO₃⁻ increases the pH, which is compensated by pulses of HNO₃ (panel B). Based on the titration curve of the growth medium, the pH interval translates into a HNO₃ concentration interval (panel C). Depending on the starting materials and microorganism selected, the culture produces N₂ and CO₂ which are released (not shown). The culture vessel may be sparged modestly with N₂.

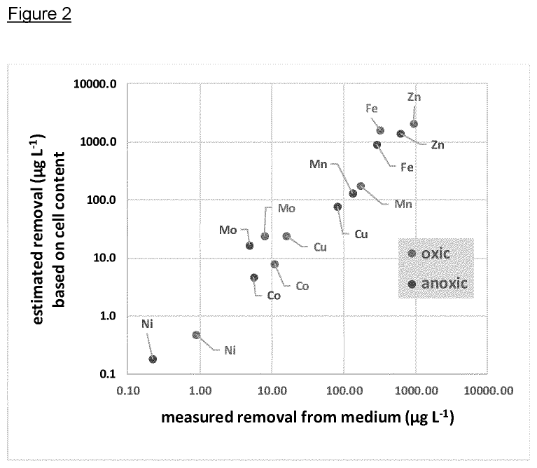

FIG. 2 shows trace metal uptake based on measured removal (Δconcentration in the liquid) and on measured trace elements in cells.

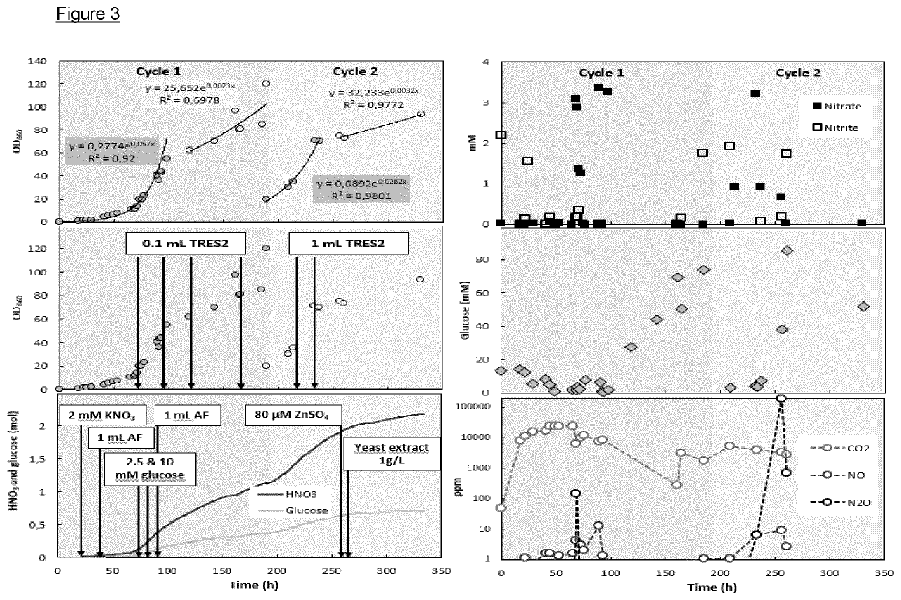

FIG. 3 shows the liquid measurements/additions and headspace measurements during a 330 h anaerobic fed-batch cultivation of P. denitrificans. The fermentation spanned two cycles. The second cycle was initiated by replacing 330 mL of the culture with sterile batch medium. Left panels Top: OD₆₆₀ and apparent μ (h⁻¹) during four phases of growth; Middle: OD₆₆₀ and additions of TRES2. Bottom: acid and glucose feed (mol cumulated), and addition of KNO₃, antifoam (AF), glucose, ZnSO₄ and yeast extract. Right panel Top: Nitrate and nitrite (mM) in cell free fermenter liquid; Middle: glucose (mM) in cell free fermenter liquid; Bottom: headspace gasses, CO₂, NO and N₂O (ppm).

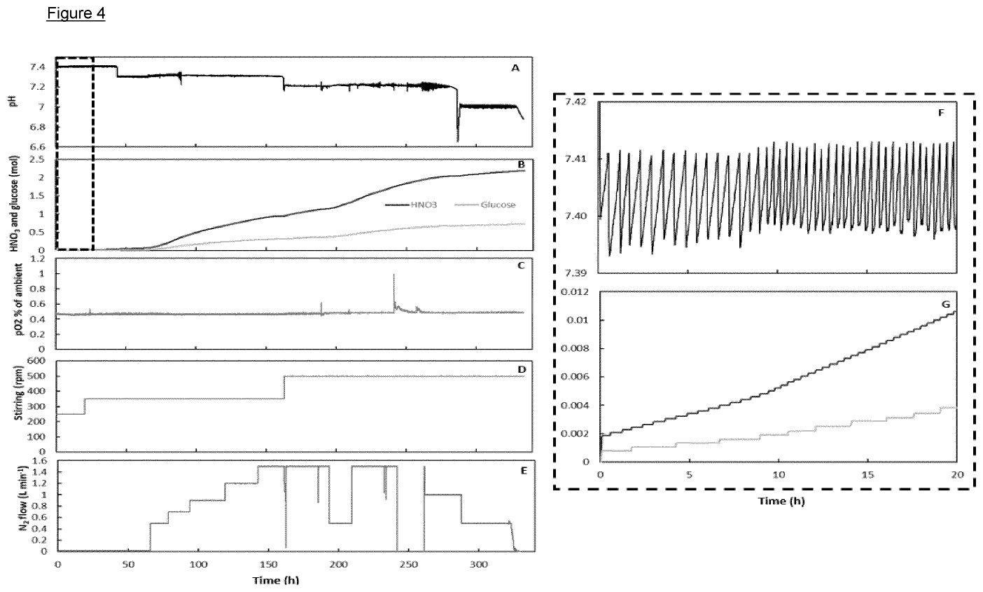

FIG. 4 provides an overview of the 330 h anaerobic fed-batch cultivation of P. denitrificans. A: continuous pH monitoring. Setpoint pH was adjusted (down) several times to increase NO₃⁻ concentration. B: HNO₃ and glucose feed (cumulated). C: pO₂, % of ambient. D: stirring speed (rpm). E: N₂ sparging through the fermenter liquid (L min⁻¹). F and G: Panels show the rise and fall in pH and the acid+glucose feed during the first 20 h of the fermentation.

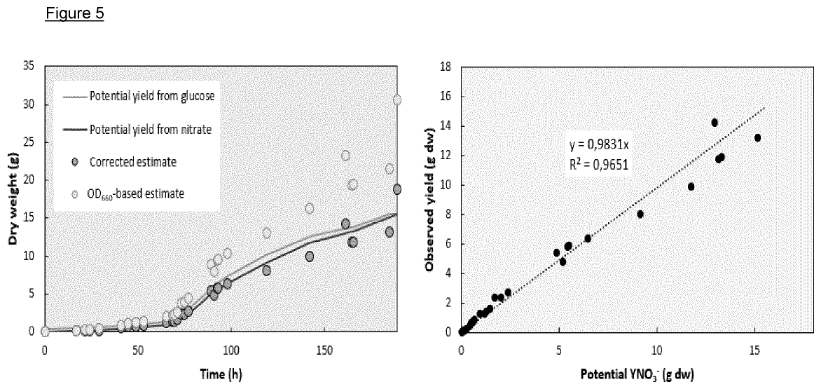

FIG. 5 shows the total dry weight yield in the bioreactor during cycle 1. Left panel: Potential yield from glucose (light grey line) and NO₃⁻ (dark grey line), direct OD₆₆₀-based estimates (light grey circles) and values corrected (dark grey circles) based on direct dry weight measurement from the endpoint of cycle 1 (189 h). Increasing colouration of cells and liquid during the fermentation was taken into account. Right panel: Reasonable fit between potential yield from NO₃⁻ reduction (YNO₃⁻) and observed dry weight.

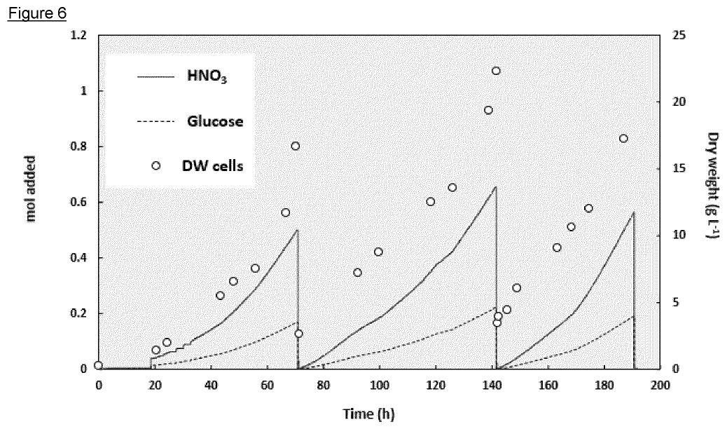

FIG. 6 shows three cycles of anaerobic culture and harvest of Paracoccus denitrificans cells for protein purification. The traces show nitric acid (mol cumulated, solid line) and glucose feed (mol cumulated, dashed line), and dry weight of cell g/L (cumulated, open circles).

### Example 1: Assessment of Culture Conditions for Paracoccus denitrificans for High Cell Density Culture

Prior to conducting high cell density growth (as discussed in Example 2), preliminary studies were carried out to optimize conditions for Paracoccus denitrificans.

Growth parameters for anaerobic growth on glucose were assessed. A series of batch incubations with glucose as the C-source, and with mineral base medium similar to that found to be optimal by Hahnke et al (2014, Front. Microbiol., 5(18), p1-9) were conducted. As a control, Sistrom's medium with succinate and ammonium, as used previously (Bergaust et al 2008, 2010, supra) was used. The glucose and nitrate concentrations were adjusted to levels that secured either nitrate- or glucose limited growth, in order to determine the growth yield per mol glucose and per mole nitrate.

The pH was adjusted to 7.5 in all media before inoculation with P. denitrificans Pd1222. The temperature was 30° C., and glucose concentrations 2.9, 5.8 and 11.6 mM were used. Cultures were first raised from frozen stocks by aerobic batch cultivation. Mid-log cultures were then used as inocula to vials with 3% O₂ in the headspace and 10 mM NO₃ in the medium. When all oxygen and NO₃ had been depleted, the cultures were used to inoculate He-washed vials with the various media, which were then monitored for the kinetics of NO, N₂O and N₂.

Cell density and yield were monitored by OD₆₆₀ measurements and dry weight determination. Dry weight was determined by harvesting cells from 35 mL cultures, by centrifugation at 10 000 rpm, 4° C. (Beckmann, JA-12 rotor), pellets were washed twice in MQ water before drying at 100° C. until they achieved a constant weight. Protein was assessed from a 10 mL culture subject to centrifugation and the pellet was stored at −20° C. until protein quantification. Gas kinetics were monitored by an incubation robot described by Molstad et al. (2007, J. Microbiol. Methods, 71 p202-211).

These cultures allowed the determination of growth kinetics and growth yield such that appropriate starting conditions for high density cell culture could be determined, i.e. optimized carbon source concentration and the timing of accumulation of undesired products (e.g. NO) and cell growth (data not shown).

A first attempt to grow P. denitrificans to high cell density by anaerobic growth was conducted in a 2 L fermenter that could be run as a “pH-stat” batch culture, where the pH was controlled by injection of 5 M HNO₃.

The fermenter experiment was started by inoculating a 1 L volume of the mineral medium, containing 10 mM NO₃⁻ and 55.6 mM (10 g/L) glucose. The OD₆₆₀ and the concentrations of nitrite, nitrate and glucose were measured at intervals, as well as the headspace concentrations of (N₂), O₂, CO₂, NO and N₂O.

To secure adequate provision of minerals in proportion to growth, the 5 M HNO₃ solution contained the same minerals as in the medium, but at 1.75 × the concentration in the mineral medium: MgSO₄*7H₂O 0.875 g/L, CaCl₂*2 H₂O 0.175 g/L; KH₂PO₄ 3.45 g/L, K₂HPO₄ 26.1 g/L; trace elements were added at the same concentrations as in the mineral medium.

Based on the empirically determined titration curve of the medium, the desired range of pH setpoints were determined (data not shown).

The experiment proved that it was possible to sustain anaerobic growth by homeostatic control of the pH by HNO₃ (data not shown). The results suggested that the oxic to anoxic transition should be avoided and that sparging with N₂ is helpful to lower the partial pressure of CO₂ (P_{CO2}) because P_{CO2} implicitly affects the NO₃ levels (high P_{CO2} lowers the pH). It was also found that it was preferable to provide have glucose, minerals and trace elements in separate reservoirs.

Trace element uptake was also examined. Cells and liquids from the cultures were analyzed by ICPMS. Cells from aerobic and anaerobic batches were centrifuged, and the supernatant taken for analysis of trace elements. The cell pellet was washed twice in H₂O by resuspension and centrifugation, then dried and digested for element analysis by ICP MS.

Trace element uptake based on the reduction of trace elements in the solution of the batch cultures, was compared with the uptake estimated by the cell dry weight and the measured trace elements in the cell dry weight. The results are shown in FIG. 2. It has also been determined that some trace elements, e.g. trace metals such as Fe²⁺ may be inhibitory on cell growth and hence the feed rate of the trace elements should take this into account.

Based on the data generated in these initial studies, appropriate media and concentrations of the carbon source, trace minerals etc. for the formulation of feed and batch media were determined for high cell density culture.

### Example 2: Growth of Paracoccus denitrificans to High Cell Density by Anaerobic Respiration Using Nitric Acid as the Terminal Electron Acceptor

Materials and Methods

Pre-Culture

P. denitrificans Pd1222 carrying a mCherry-nirS gene fusion replacing the native nirS (Lycus et al., 2018, PNAS, 115(46), p11820-11825) was raised from glycerol stock aerobically overnight at 30° C. The mCherry fusion tag enables the visual inspection of NirS expression through fluorescence microscopy but is otherwise of no consequence to the denitrification phenotype. The exponentially growing aerobic culture was used as inoculum (˜2.3×10⁸ cells added) in sealed 120 mL vials containing approximately 1% O₂ in the headspace, 10 mM glucose and 5 mM NO₃⁻ (provided as KNO₃) in 50 mL medium. All cultures were stirred at approximately 700 rpm to ensure sufficient gas exchange between the headspace and liquid. The anaerobic cultures were monitored in the incubation system described in Molstad et al. (2007, J. Microbiol. Methods, 71, p202-211) and additional NO₃ in increments of 5 mM was added (provided as KNO₃) when needed. A total volume of 45 mL anoxic culture (˜1×10¹ cells) was used as the inoculum to start the fed-batch culture. The composition of the medium, trace elements solution, glucose- and HNO₃ feed are shown in Tables 1 and 2. Trace elements solution (TRES2) was injected (0.1 mL and 1 mL) several times during the fermentation (FIG. 3), guided by the cell density.

TABLE 1
Composition of start-up medium and trace elements solution.

| | In start-up medium | TRES2 (5000x) |
|---|---|---|
| Component | M | M |
| EDTA | 5.25 × 10⁻⁶ | |
| ZnSO₄*7H₂O | 3.81 × 10⁻⁵ | 4.45 × 10⁻³ |
| FeSO₄*7H₂O | 1.80 × 10⁻⁵ | 2.00 × 10⁻¹ |
| MnSO₄ *H₂O | 9.11 × 10⁻⁶ | 3.29 × 10⁻² |
| CuSO₄* 5H₂O | 1.60 × 10⁻⁶ | 1.51 × 10⁻² |
| Co(NO₃)₂*6H₂O | 1.01 × 10⁻⁶ | 9.28 × 10⁻⁴ |
| H₃BO₃ | 1.84 × 10⁻⁶ | 2.99 × 10⁻⁴ |
| Na₂MoO₄*2H₂O | 1.00 × 10⁻⁶ | 1.87 × 10⁻³ |
| NiCl*6H₂O | | 2.34 × 10⁻⁵ |
| MgSO₄ | 1.69 × 10⁻² | |
| CaCl₂*2H₂O | 4.63 × 10⁻³ | |
| KH₂PO₄ | 7.24 × 10⁻³ | |
| K₂HPO₄ | 4.28 × 10⁻² | |

TABLE 2
Liquid reservoirs connected to the bioreactor. pH was monitored
continuously, and acid was fed to the culture as a response
to pH increase, whereas glucose was fed proportionally (k =
0.534) to the acid. Trace elements (TRES2) were added manually.

| | Component | Conc. (M) |
|---|---|---|
| Acid | HNO₃ | 5 |
| | MgSO₄ | 1.28 × 10⁻² |
| | CaCl₂*2H₂O | 5.25 × 10⁻³ |
| | K₂HPO₄ | 8.86 × 10⁻³ |
| | NaH₂PO₄ | 1.29 × 10⁻² |
| Antifoam (Glanapon 2000) | Antifoaming agent | * |
| Feed | Glucose | 3.125 |

* Added to final concentration of 1 mL L⁻¹ in reactor

The Anoxic Fed-Batch Culture

The bioreactor was set up with 500 mL sterile medium (Table 1) containing 10 mM glucose. The temperature was adjusted to 30° C. and the bioreactor was sparged with N₂ to remove O₂ before inoculation. The anaerobic pre-culture (45 mL) was added through a septum in the top plate and the acid pump initialized by adjusting the pH setpoint to 7.4. Liquid- and gas samples were taken regularly throughout the fermentation. The fermentation ran for approximately 330 h with adjustments made to pH setpoint (to increase acid concentration), stirring, and gas flow (to counteract pH drop due to CO₂ production) (FIG. 4). The supply of electron acceptor and nitrogen (HNO₃), electron donor and carbon source (glucose) was controlled by the metabolism of the culture: as nitrate was taken up, pH increased, triggering the acid pump, which in turn triggered the feed pump (FIGS. 4, F and G).

Additional glucose was added twice during the first 100 h to ensure a slight excess because C-limitation increases the risk of high NO accumulation (data not shown).

Results

The culture grew exponentially to high density as seen by the increase in OD₆₆₀ during the first 100 hours (FIG. 3; μ=0.057 h⁻¹). Glucose, nitrite and nitrite were kept in the low mM range, although after 100 h, some glucose accumulated in the fermenter. NO and N₂O were with few exceptions kept below 10 ppm and below detection (<2 ppm), respectively, whereas CO₂ accumulated (maximum 23800 ppm in headspace) (FIG. 3), and N₂-flow was adjusted several times during the fermentation to counteract excessive CO₂ accumulation (FIG. 4, E).

Beyond 100 h, the growth rate decreased although the culture continued to grow to a maximum observed OD₆₆₀ of 120. Several attempts were made to boost growth: addition of trace elements (TRES2) and glucose during the first 189 h (“cycle 1”), and addition of high amounts of TRES2, Zn and yeast extract during the last ˜150 h of the cultivation (“cycle 2”). After 189 h, most of the fermenter liquid was extracted and replaced by fresh medium (cycle 1→cycle 2). This resulted in a temporary increase in growth rate, although impaired compared to the initial phase of the fermentation. Approximately 50 h into cycle 2, the growth rate again decreased dramatically and remained low until the end of the experiment (FIG. 3). Subsequent studies suggest this is due to excess trace elements, particularly Fe²⁺ which should be controlled to avoid detrimental effects.

During the two periods with the highest observed growth rate (0-100 h and 189-240 h), glucose was kept in the low mM range and did not exceed 20 mM. However, declines in growth rates were accompanied by a gradual increase in glucose concentration, up to maxima of 74 mM towards the end of cycle 1, and 85 mM midway in cycle 2 (261 h). At this point, N₂O accumulated to extremely high concentration.

The apparent cell yield based on OD₆₆₀ was considerably higher than would be expected from the total amounts of nitrate and glucose added to the bioreactor. This was partly due to a gradually deepening brownish coloring of the culture, which skewed the OD₆₆₀ measurements. However, at the end of cycle 1, samples were collected for dry weight determination. This was used to correct for the overestimation due to the coloration of the cells and medium, and the adjusted yields fit reasonably well with the amount of glucose and nitrate supplied during the fermentation (FIG. 5).

### Example 3: Multi-Cycle Growth of Paracoccus denitrificans to High Cell Density by Anaerobic Respiration Using Nitric Acid as the Terminal Electron Acceptor

Materials and Methods

Culture was similar to the methods used in Example 2 in the anoxic batch-fed culture. Three cycles of anaerobic culture and harvest of Paracoccus denitrificans cells for protein purification were completed. Paracoccus denitrificans was cultured under denitrifying conditions with glucose as carbon- and e⁻ donor in a pH stat where HNO₃ served to control pH and was also the sole nitrogen and e⁻ acceptor. The target cell density was 15-25 g dry weight L⁻¹. For each cycle, once the culture reached the preferred density, ˜80% of the culture volume was harvested and replaced with sterile medium, before HNO₃ and glucose feeding resumed.

Results

The results are shown in FIG. 6. Culture to high density was readily achieved without loss of yield. Dry weight yields in excess of 10 g/L were achieved. There is nothing to suggest that higher yields could not be achieved as no signs of growth inhibition were observed even at the high densities that were achieved.

1. A method for anaerobic culture of denitrifying microorganisms in culture medium, comprising the addition of nitric acid and/or nitrous acid two or more times to said medium during said culture, wherein the pH of the medium is maintained within a pH interval by the addition of the nitric acid and/or nitrous acid and said nitric acid and/or nitrous acid also provides the electron acceptor for denitrification in said culture.

2. The method as claimed in claim 1, wherein the denitrifying microorganisms are bacteria, preferably Paracoccus denitrificans.

3. The method as claimed in claim 1 or 2, wherein said nitric acid and/or nitrous acid is added ten or more times to said medium during said culture and/or at least every hour during said culture.

4. The method as claimed in claim 3, wherein said nitric acid and/or nitrous acid is added to said medium continuously during said culture.

5. The method as claimed in any one of claims 1 to 4 wherein the concentration of the nitric acid and/or nitrous acid which is added to the culture medium is ≥2M.

6. The method as claimed in any one of claims 1 to 5 wherein the initial concentration of the nitric acid and/or nitrous acid in the culture at the point of each addition is from 2 to 10 mM.

7. The method as claimed in any one of claims 1 to 6, wherein the nitric acid and/or nitrous acid is added to said medium when the pH of the medium rises above the midrange of the pH interval, or when the pH rises to the upper level of the pH interval.

8. The method as claimed in claim 7, wherein the pH of the culture medium is monitored and nitric acid and/or nitrous acid is added when the pH of the medium rises above the midrange of the pH interval, or when the pH rises to the upper level of the pH interval.

9. The method as claimed in any one of claims 1 to 8, wherein the pH interval is ≤1 pH unit, preferably ≤0.5, 0.4, 0.2, 0.1 or 0.05 pH unit.

10. The method as claimed in any one of claims 1 to 9, wherein the midrange of the pH interval is within 0.4 pH units of the microorganism's optimal pH range, preferably in the range of pH 7 to 8.

11. The method as claimed in any one of claims 1 to 10 wherein no nitrate salts are used in said method except in the initial starting culture.

12. The method as claimed in any one of claims 1 to 11 wherein said nitric acid and/or nitrous acid provides at least 90% of the nitrate to the culture.

13. The method as claimed in any one of claims 1 to 12, wherein the culture is performed for at least 50 hours, preferably at least 100, 200 or 300 hours.

14. The method as claimed in any one of claims 1 to 13, wherein the microorganisms in the culture are grown to a cell density of at least 3 g cell dry weight L⁻¹.

15. The method as claimed in any one of claims 1 to 14, wherein the microorganisms in the culture are grown to a high cell density, preferably at least 1×10¹⁴ cells L⁻¹ or at least 10 g cell dry weight L⁻¹.

16. The method as claimed in any one of claims 1 to 15, wherein the salt concentration in said culture resulting from the addition of nitrate to said culture is less than 100 mM.

17. The method as claimed in any one of claims 1 to 16, wherein said denitrifying microorganism is a heterotrophic microorganism, wherein preferably the carbon source and electron donor for said culture is provided by a saccharide or the base of an organic acid, preferably glucose.

18. The method as claimed in any one of claims 1 to 16, wherein said denitrifying microorganism is an autotrophic microorganism, wherein preferably the carbon source for said culture is provided by CO₂ and the electron donor for said culture is provided by hydrogen.

19. The method as claimed in any one of claims 1 to 17, wherein substantially all CO₂ is removed from the culture headspace during the culture method.

20. The method as claimed in any one of claims 1 to 19, wherein the culture medium contains a carbon source.

21. The method as claimed in any one of claims 1 to 20, wherein the carbon source and/or trace elements are added to the medium two or more times during said culture, wherein preferably the carbon source and trace elements are added separately.

22. The method as claimed in any one of claims 1 to 21, wherein during said culture, the carbon source is added to the medium in an amount and at a time, relative to the amount and timing of the nitric acid/nitrous acid additions, so that the concentration of the carbon source is not rate-limiting on growth of the microorganism.

23. The method as claimed in any one of claims 1 to 22 wherein the nitric acid and/or nitrous acid is the rate-limiting component in the culture method.

24. The method as claimed in any one of claims 1 to 23, wherein the culture is fed-batch, repeated fed-batch or continuous.

25. A method for producing a cell culture product comprising:
a) culturing denitrifying microorganisms by anaerobic culture using a method as defined in any one of claims 1 to 24 under conditions that allow the generation of said cell culture product; and
b) obtaining said cell culture product from said culture.

26. The method as claimed in claim 25, wherein said cell culture product is cultured cells.

27. The method as claimed in claim 25, wherein said cell culture product is a molecule produced by said microorganisms, preferably a protein.

28. The method as claimed in any one of claims 25 to 27, wherein said step of obtaining the product comprises separating said product from the medium and/or microorganisms and optionally further purifying said product.

29. The method as claimed in any one of claims 25 to 28, wherein said method additionally comprises the step of processing the obtained cell culture product to provide a processed cell culture product, preferably by drying the obtained cell culture product or formulating a composition comprising the obtained cell culture product.

30. The method as claimed in claim 29, wherein the processed cell culture product is single cell protein, feed or a pharmaceutical composition.

31. A cell culture product or processed cell culture product obtained by a method as defined in any one of claims 25 to 30.
